(19) United States
(12) Patent Application Publication
(10) Pub. No.: US 2007/0055320 A1
(43) Pub. Date: Mar. 8, 2007

(54) Methods for treating temporal lobe epilepsy, associated neurological disorders, and other patient functions

(75) Inventor: Martin E. Weinand, Tucson, AZ (US)

Correspondence Address: PERKINS COIE LLP;PATENT-SEA, P.O. BOX 1247, SEATTLE, WA, 98111-1247, US

(73) Assignee: Northstar Neuroscience, Inc., Seattle, WA (US)

(21) Appl. No.: 11/518,139

(22) Filed: Sep. 7, 2006

Related U.S. Application Data
(60) Provisional application No. 60/714,705, filed on Sep. 7, 2005.

(51) Int. Cl. A61N 1/00

(52) U.S. Cl. 607/45

(57) ABSTRACT

Methods for treating temporal lobe epilepsy, associated neurological disorders, and other patient functions are disclosed. Methods for treating patients in accordance with several embodiments include implanting a signal delivery device subdurally proximate to a target neural site at a cortical location of a patient. The method can further include applying electrical signals to the target neural site via the signal delivery device, on a generally continual basis at a frequency of from about 0.9 Hz to about 250 Hz. The electrical signals can be applied to epileptic patients to at least reduce ictal and interictal epileptic senicity, and/or to patients functioning at normal or better levels to improve patient functioning.

### CROSS-REFERENCE TO RELATED APPLICATIONS

The present application claims the benefit of U.S. Provisional Application No. 60/714,705, filed Sep. 7, 2005, and incorporated herein by reference.

### TECHNICAL FIELD

The present invention relates generally to methods for treating temporal lobe epilepsy, associated neurological disorders, and/or other patient functions, for example, with an implantable cortical stimulation device.

### BACKGROUND

Approximately 0.5 to 1% of the United States population has epilepsy, defined as recurrent seizures. Epilepsy results from the abnormal, excessive discharge of neurons in the brain producing recurrent seizures. Among patients with epilepsy, approximately 20% of all patients are medically intractable, meaning that medication which is designed to control the patient's seizures is not satisfactorily effective (Surgery for Epilepsy, NIH Consensus Development Conference Statement, Mar. 19-21, 1990, http://consensus.nih.gov/cons/077/077_statement.htm). Temporal lobe epilepsy is one of the most difficult forms of epilepsy to control with medication. Therefore, temporal lobectomy is the most commonly performed resective brain procedure designed to treat medically intractable epilepsy (Weinand et al., Journal of Neurosurgery 86: 226-232, 1997). Seizures originating from the temporal lobe in patients who are medically intractable most often begin in medial temporal lobe structures (Weinand et al., Journal of Neurosurgery 77: 20-28, 1992), including the hippocampus and amygdala. While approximately 65% of patients undergoing temporal lobectomy will be rendered seizure-free (Weinand et al., Seizure 3: 55-59, 1994), many patients must remain on antiepileptic medication associated with cognitive and other side effects. In addition, the temporal lobectomy operation itself can pose significant risks including stroke (e.g., hemiparesis and/or aphasia).

Selection of patients for temporal lobectomy may be difficult and/or time consuming in that the process may involve long-term video-scalp EEG monitoring, MRI brain scans, Positron Emission Tomography (PET) or Single Photon Emission Computed Tomography (SPECT) scans, surface cortical cerebral blood flow monitoring, neuropsychological testing and intracarotid amytal testing to localize the epileptic focus for eventual resection. For patients in whom non-invasive or minimally invasive localization of the seizure focus fails, invasive EEG monitoring with subdural strip electrodes may be necessary (Weinand et al., Journal of Neurosurgery 77: 20-28, 1992). Accordingly, there is a need for improved methods and systems for addressing temporal lobe epilepsy. There is also a need for addressing other patient functions, associated with and/or independent of epilepsy.

### BRIEF DESCRIPTION OF THE DRAWINGS

FIG. 1 illustrates a patient with an implanted pulse generator and signal delivery device configured in accordance with an embodiment of the invention.

FIGS. 2A and 2B illustrate multiple electrode devices implanted in a patient in accordance with an embodiment of the invention.

FIG. 3 is a flow diagram illustrating a process for treating a patient in accordance with several embodiments of the invention.

FIG. 4 is a schematic illustration of a pulse system configured in accordance with several embodiments of the invention.

FIG. 5 is an isometric view of an electrode device that carries multiple electrodes in accordance with another embodiment of the invention.

### DETAILED DESCRIPTION

The present disclosure is directed generally to methods for treating temporal lobe epilepsy, associated neurological disorders, and other patient functions. Such methods can be used to reduce the occurrence of epileptic seizures (e.g., ictal events) and other disorders or dysfunctions that are associated with epilepsy, but that occur in between seizures (e.g., interictal events). For example, a particular method includes identifying a target neural site at a parahippocampal gyrus of a patient. The method can further include implanting an electrode at a subdural location at least proximate to the target neural site, and at least reducing both ictal and interictal epileptogenicity of the patient, including interictal neural dysfunction associated with epileptogenicity. In particular embodiments, at least reducing interictal neural dysfunction associated with epileptogenicity can include at least reducing the effects of a neuropsychological or neuropsychiatric disorder. Such disorders can include an interictal behavior syndrome disorder, depression, an obsessive-compulsive disorder, rage attacks, an anxiety disorder, a disassociative disorder, or an experiential disorder, among others.

Another method for treating a patient can include implanting an electrical signal delivery device proximate to a target neural site located at a temporal lobe of the patient. The method can further include at least reducing non-epileptogenic symptoms of the patient by applying electrical signals to the target neural site via the electrical signal delivery device on a generally continual basis at a frequency of from about 0.9 Hz to about 250 Hz. In particular embodiments, at least reducing non-epileptogenic symptoms can include reducing the symptoms associated with a stroke, tinnitus, neuropsychological disorders, and/or neuropsychiatric disorders, among others.

Still another method for treating a patient includes implanting an electrical signal delivery device proximate to a target neural site at a cortical location of the patient, and improving a non-epileptogenic neurological characteristic of the patient by applying electrical signals to the target neural site. The electrical signals are applied via the electrical signal delivery device on a generally continual basis (e.g., greater than 90% of the time) at a frequency of about 0.9 Hz to about 250 Hz. In particular embodiments, improving the neurological characteristic of the patient can include improving a neurological characteristic of the patient that is at normal (or better) levels. For example, the technique can include improving a memory function of the patient, a neuropsychological function of a patient, or another function of the patient. In other particular embodiments, the method can further include directing the patient to engage in an adjunctive behavior as part of a treatment regimen that includes both the adjunctive behavior and the application of electrical signals. For example, the adjunctive behavior can include engaging the patient in a language task, a memory task, a musical task, a mathematical task, or another task that relates to the function that is enhanced by the application of electrical signals.

Still another method for treating a patient includes identifying a target cortical neural site associated with a function that the patient performs at normal (or better) levels. The method can further include implanting an electrical signal delivery device at a subdural location proximate to the target cortical neural site, and improving the function of the patient by applying electrical signals to the target neural site via the electrical signal delivery device on a generally continual basis at a frequency of from about 0.9 Hz to about 250 Hz. The method can still further include receiving automated feedback from the patient corresponding to the function, and changing a location at which signals are provided, based at least in part on the automated feedback. For example, the electrical signal delivery device can include multiple electrodes or contacts, and changing a location at which the signals are provided can include changing a contact to which the signals are directed.

Still further aspects relate to the use of subdural strip electrodes. In one embodiment, an electrode device generally similar to a diagnostic EEG recording device can operate as a permanently implanted therapeutic conduit for long-term cerebral cortical stimulation to suppress and treat temporal lobe epileptogenicity and associated neurological disorders. The device can optionally also provide an EEG or other diagnostic function and/or can be used to enhance patient functions in addition to or in lieu of treating epileptogenicity.

The medial temporal lobe includes brain structures that are responsible for a diversity of significant, eloquent brain functions. Within the medial temporal lobe are the hippocampus and amygdala, brain structures that are components of the limbic system, a part of the brain substantially involved in normal human central nervous system function and disease. The medial temporal lobe is highly epileptogenic, that is, prone to developing epilepsy (Weinand et al., Journal of Neurosurgery 77: 20-28, 1992). Patients with temporal lobe epilepsy may suffer from the effects of the interictal behavior syndrome disorder of temporal lobe epilepsy (Waxman S G, Geschwind N, Arch. Gen. Psychiatry 1975 December; 32(12):1580-6.) which may be responsive to therapeutic subdural cortical stimulation and include alterations in sexual behavior, religiosity, and a tendency toward extensive, and in some cases compulsive, writing and drawing. Symptoms of this temporal lobe syndrome may also include disturbed language, learning, memory and behavior, behavioral changes including episodic mood disorders, hyperirritability, anger, and aggressive outbursts, and in cases of dominant temporal lobe epilepsy, impairment of language (aphasias) and disorders of sensation and sensory integration (The American Psychiatric Textbook of Neuropsychiatry, 3d ed, S C Yudofsky and R E Hale, eds. Washington, D.C., APA, 1997 and in Marion D W. Traumatic Brain Injury. New York, Thieme, 1999).

In the Epilepsy Spectrum Disorder (ESD) (http://subtlebraininjury.com/seizure.html), patients with temporal lobe epilepsy may experience one or more of the following symptoms which may be responsive to therapeutic subdural cortical stimulation therapy: memory gaps, confusional spells, staring spells, episodic irritability, episodic tinnitus, episodic aphasia, jamais vu, olfactory hallucinations, gustatory hallucinations, visual illusions (e.g., scintillations), paresthesias, anesthesias, auditory illusions (e.g., phone ringing), headache with nausea and/or photophobia, abrupt mood shifts, déjà vu, “odd” abdominal sensations, intrusive thoughts and parasomnias. In addition, the medial temporal lobe contains structures that are or may be involved with normal health and the pathophysiology of brain disorders associated with or related to epilepsy, and which may be responsive to subdural cortical stimulation. Representative disorders include memory disorders (i.e., for old and/or new verbal and/or nonverbal material, as in such disorders as Alzheimer's disease and post-traumatic amnesia), emotional and affective health and disease (i.e., depression, anxiety, rage attacks, agoraphobia) and, by adjacent association cortex, the synthesis, interpretation and expression of multiple sensory functions including vision, hearing, olfaction and complex integrative functions such as sensory-memory and motor-memory.

In some patients with temporal lobe epilepsy, epileptic foci may be localized to the lateral temporal cortex. In a regional (medial and lateral) temporal lobe distribution requiring therapy designed to treat (i.e., resect or neuromodulate) portions of the temporal lobe including and in addition to the medial temporal lobe (i.e., lateral and regional temporal cortex), aspects of the invention may be tailored to accommodate knowledge of a specific patient's temporal lobe seizure focus in order to optimize or at least enhance seizure control and treat associated neurological dysfunction. Using therapeutic subdural strip electrode stimulation as an example for seizure focus treatment, the appropriate area of the temporal lobe cortex (i.e., medial, lateral or medial and lateral) may be electrically stimulated to produce an effective therapeutic response. In addition, by virtue of secondary epileptogenesis and/or association with multiple epileptic foci, some patients with temporal lobe epilepsy may have extratemporal (i.e., frontal, parietal and/or occipital) lobe epilepsy and may require treatment (i.e., therapeutic cortical stimulation) in addition to medication to control intractable epilepsy. In designing and implementing a therapeutic paradigm for cortical stimulation to treat intractable seizures, additional benefits beyond seizure control may include treatment of neuropsychological and sensory-motor disorders using subdural cortical stimulation, including symptoms which may be associated with epilepsy such as motor, sensory, visual, receptive and/or expressive language, memory, olfactory and/or auditory dysfunction and neuropsychological or psychiatric disorders such as depression, obsessive-compulsive disorder, rage attacks, anxiety disorders and disassociative/experiential states and disorders, depending upon the location of the cerebral cortex dysfunction that is amenable to stimulation and neuromodulation. As is discussed in greater detail later, these and other disorders or dysfunctions may also be treated in a patient independently of whether the patient is epileptogenic or not.

Temporal lobe cortical stimulation may be therapeutic for the interictal behavior syndrome of temporal epilepsy, and may improve functioning in areas including disorders in sexual behavior, religiosity, and a tendency toward extensive, and in some cases compulsive, writing and drawing. Temporal lobe cortical stimulation therapy may, while treating temporal lobe epilepsy, be therapeutic for the associated temporal lobe syndrome and signs and symptoms of the temporal lobe syndrome (i.e., symptoms of disturbed language, learning, memory and behavior, behavioral changes including episodic mood disorders, hyperirritability, anger, and aggressive outbursts, and in cases of dominant temporal lobe epilepsy, impairment of language (aphasias) and disorders of sensation and sensory integration). Temporal lobe cortical stimulation may be therapeutic for the psychiatric symptoms characteristic of the neurobehavioral syndrome of epilepsy (i.e., Morel syndrome) (http://www.emedicine.com/neuro/topic604.htm). These symptoms include psychotic or other psychiatric symptoms, including interictal and/or post-ictal psychosis; depression or elation, or an anxiety syndrome; paranoid delusions and delusions of reference; mood disorders with particular emphasis on depression (seen particularly in temporal lobe epilepsy and in up to 55% of patients with epilepsy); the peri-ictal prodrome seen in up to 20% of patients with epilepsy consisting of a depressed-irritable mood, sometimes with anxiety or tension and headaches, and including symptoms of depressed mood, anergia, pain, insomnia, fear and/or anxiety; an increased risk of suicide (as high as 13%, 5 to 10 times the normal population incidence); mania; anxiety disorders (ranging from 19 to 57% in epilepsy patients); personality disorders; and Geschwind syndrome, associated particularly with temporal lobe epilepsy, consisting of viscosity, circumstantiality, hypergraphia, and hyperreligiosity (http://www.emedicine.com/neuro/topic604.htm).

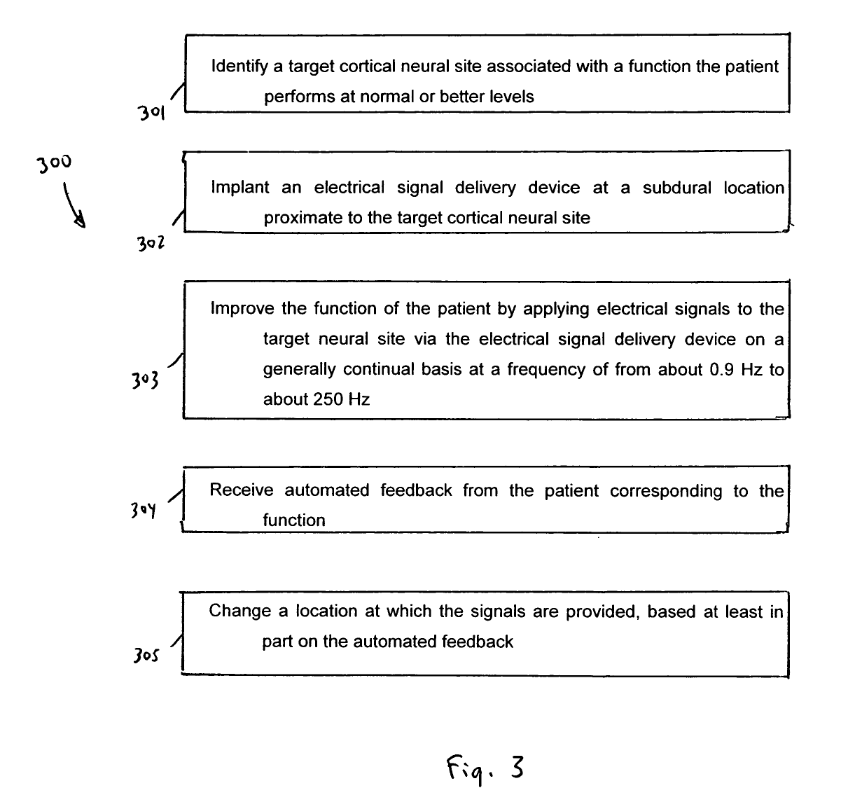

In addition to treating epilepsy and associated neurological and neuropsychological disorders, cortical stimulation for temporal and/or extratemporal lobe epilepsy can enhance normal (and/or above normal) functioning of the brain, including accentuating memory, language, visual, sensory, motor, auditory, gustatory and olfactory functions as well as complex integration of two or more of these eloquent functions. By improving normal (and/or above normal) brain function via cortical stimulation, complex multidimensional sensory-motor, mental and emotional functions may be accentuated, thereby improving the overall quality of life, pleasure, efficiency, time-processing capabilities, verbal and nonverbal intelligence and creativity associated with enhanced human brain function. Further details of particular methods for enhancing normal or above normal functions are discussed later with reference to FIG. 3.

Cortical stimulation may inhibit interictal and ictal temporal lobe and/or extratemporal lobe epileptogenicity. The epileptic temporal lobe is hypoperfused during the interictal state (Weinand et al., Journal of Neurosurgery 86: 226-232, 1997). The efficacy of the therapeutic response to cortical stimulation may involve normalization of cerebral blood flow which may return from the interictal epileptic, hypoperfused state to normal cerebral blood flow during properly positioned and configured cortical stimulation. Normalization of cerebral blood flow from a baseline hypoperfused state, has also been shown to produce profound improvements in general cognitive functioning including a reversal of a coma state to normal consciousness (Sioutos P, Orozco J, Carter L P, Weinand M E et al., Neurosurgery 36: 943-949, 1995). Normalization of cerebral blood flow in temporal lobe epilepsy is associated with reduction of temporal lobe epileptogenicity (Weinand et al., Journal of Neurosurgery 86: 226-232, 1997). Accordingly, in at least one embodiment, it is expected that effective treatment of temporal lobe epilepsy and its associated neurological disorders is due to improved cerebral blood flow provided by electromagnetic stimulation. It is further expected that, when maintained below the cortical threshold for neuronal after-discharge, cortical stimulation in humans is safe and may be efficacious over the long-term (months to years to the patient's lifetime) in the suppression of temporal and/or extratemporal lobe epileptogenicity and associated disorders of brain function.

A device configured in accordance with one embodiment of the invention includes a plastic strip (e.g., a support member or substrate) that is approximately 0.5 to 1.0 mm in thickness, approximately 0.7 to 1.0 centimeter in width and contains from 4 to 8 stainless steel contacts shaped in flat disc form and separated by approximately 0.5 to 1.0 centimeter from disc center to disc center. In patients with temporal lobe epilepsy, a burr hole approximately 1.5 centimeters in diameter can be placed in the skull in the squamous temporal bone approximately 3.0 to 4.0 centimeters superior to the zygoma. The dura can be incised twice, once horizontally in the center of the burr hole exposure and a second time vertically in the center of the burr hole exposure. To facilitate adherence of the subdural strip to the dura, various leaves of dura may remain un-incised to permit suturing of the strip lead to the dura using 4-0 silk suture to enhance the stability of the permanent implantation of the device. In most patients with temporal lobe epilepsy, at least one single four contact subdural strip electrode may be placed inferior-medially, in the subdural space over the temporal lobe, including contact between the temporal cerebral cortex and the stainless steel discs over the middle and inferior temporal, fusiform and parahippocampal gyri. The entorhinal cortex, responsible for integration of multiple sensory modalities, may also be affected by this temporal lobe cortical stimulating device. This configuration permits cortical stimulation in brain regions adjacent to the seizure focus responsible for intractable temporal lobe epilepsy.

Placing the electrode strip (or other electrical signal delivery device) at a subdural location is expected to produce one or more of several advantages in at least some embodiments. Such advantages can include relatively low power requirements (compared to an epidural location), and therefore longer battery life for the source supplying power to the electrodes. The subdural location may also produce fewer side effects. In addition, the parahippocampal gyrus is generally inaccessible via an epidural implantation. Accordingly, the subdural location can provide increased efficacy by more directly targeting the desired neural population(s). As a result, subdural electrodes can be used to the exclusion of penetrating or deep brain electrodes in at least some embodiments.

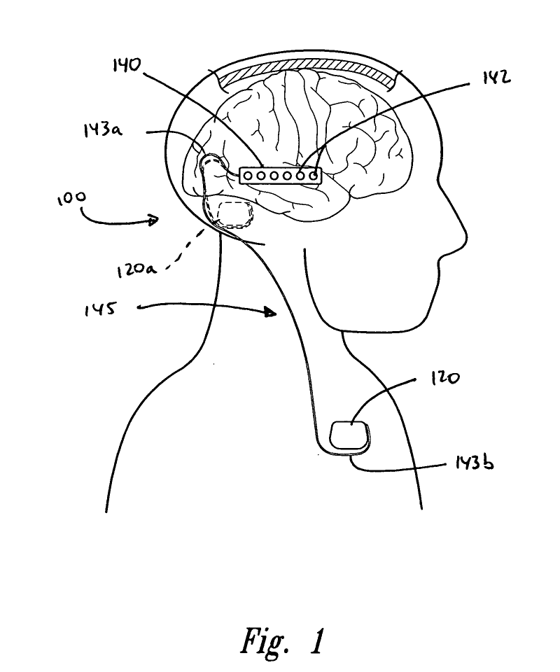

FIG. 1 illustrates a system 100 that includes an electrical signal delivery device 140 (e.g., an electrode strip), a pulse system 120 (e.g., an implantable pulse generator or IPG), and a communication link 145 that connects the pulse system 120 and the signal delivery device 140. The signal delivery device 140 can include multiple contacts or electrodes 142, six of which are shown in FIG. 1 for purposes of illustration. In other embodiments, the device 140 can include more or fewer contacts 142. In one embodiment, the communication link 145 includes a subdural strip lead that is tunneled subcutaneously above and behind the ear on the side of the head and brain containing the seizure focus. A strain relieving loop 143a in the subdural strip lead is placed above the ear. The lead is further tunneled to an incision in the ipsilateral infraclavicular location in the upper chest where the lead is attached to the pulse system 120. Another strain relieving loop 143b can be placed in the lead as it enters the pulse system 120. Cortical stimulation and cerebral blood flow data (Weinand ME, unpublished data, Oct. 11, 1999) suggest that an efficacious location for cortical stimulation in intractable epilepsy is that region of cortex most proximate to the epileptic focus. Therefore, in most patients with medial temporal lobe epilepsy, an efficacious cortical stimulation location is expected to be the parahippocampal gyrus with electrical stimulation emanating from the most distal contact 142 in the subdural strip electrode array. Representative signal delivery devices 140 and IPGs are available from Advanced Neuromodulation Systems, Inc. of Plano, Tex.

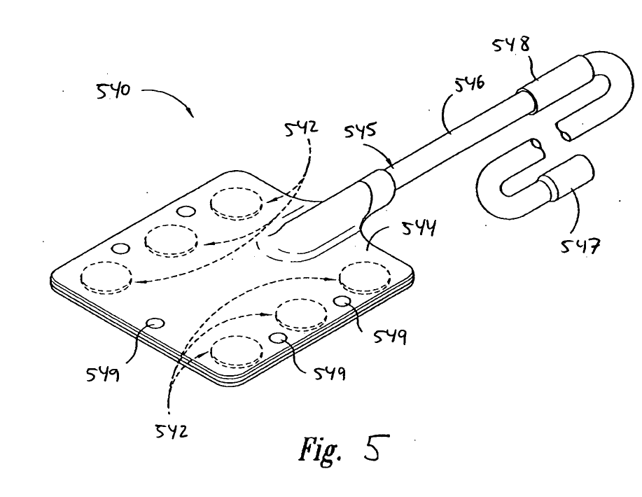

In other embodiments, the system 100 can have other configurations. For example, the system 100 can include a different pulse system 120a (shown in dashed lines in FIG. 1), which is implanted above the neck rather than below the neck. In still further embodiments, the pulse system 120 can have other configurations, for example, a shallow “can” or other housing that is inserted into a hole in the skull. In other embodiments, the signal delivery device 140 can have other configurations, for example, a grid configuration, as is discussed later with reference to FIG. 5. The signal delivery device 140 can be positioned at locations other than the medial temporal lobe, depending upon the particular dysfunction or function that it is intended to address. For example, the signal delivery device 140 can be positioned at the frontal lobe to address motor functions, the parietal lobe to address sensory functions, or the occipital lobe to address visual functions.

In one aspect of an embodiment shown in FIG. 1, the system 100 can be tailored and/or programmed to neuromodulate and treat intractable medial temporal lobe epilepsy, suppressing recurrent seizures and interictal epileptic phenomena. Such phenomena can include neurological, psychological and neuropsychiatric dysfunction associated with temporal lobe epilepsy, including the temporal lobe syndrome, the Epilepsy Spectrum Disorder (ESD), the psychiatric symptoms characteristic of the neurobehavioral syndrome of epilepsy (i.e., Morel syndrome), Geschwind syndrome, and the interictal behavior syndrome of temporal lobe epilepsy, the signs and symptoms of which are described briefly herein.

Cortical stimulation parameters programmed into the pulse system 120 for delivery by the signal delivery device 140 to the temporal cerebral cortex can vary depending on the individual patient's therapeutic response (i.e., reduction in seizure frequency and/or severity, and/or improvement in neuropsychiatric signs and/or symptoms). In general, the applied signals are subthreshold for temporal lobe after-discharges and can have signal parameters including, but not limited to, the following: a frequency of from about 0.9 to about 250 Hertz (Hz), a current of from about 0.1 to about 10 milliamperes (mA), and a pulse width of from about 0.25 to about 0.5 milliseconds (ms) using biphasic and/or alternating square waves for durations of up to 24 hours per day. In particular embodiments, the frequency can have a range of from about 0.9 Hz to about 130 Hz, or from about 50 Hz to about 100 Hz. In a further particular embodiment, the frequency can have a value of about 130 Hz.

In other embodiments, the signal delivery parameters described above can have other characteristics. For example, the pulses can have shapes other than square waves. In a particular embodiment, the signals are delivered to the patient on a generally continual basis. As used herein, the phrase generally continual refers to signals that are delivered to the patient at least 90% of the time. For example, signals can be delivered at a frequency of from about 0.9 Hz to about 250 Hz for 23 hours per day (e.g., with a one hour break per day), every day, for a period of months or years. The signal frequency can be varied within the above range during the course of signal delivery. For example, the frequency can be varied to prevent (or reduce the likelihood of) patient habituation. The frequency may at times be reduced (within the above range) to conserve system power.

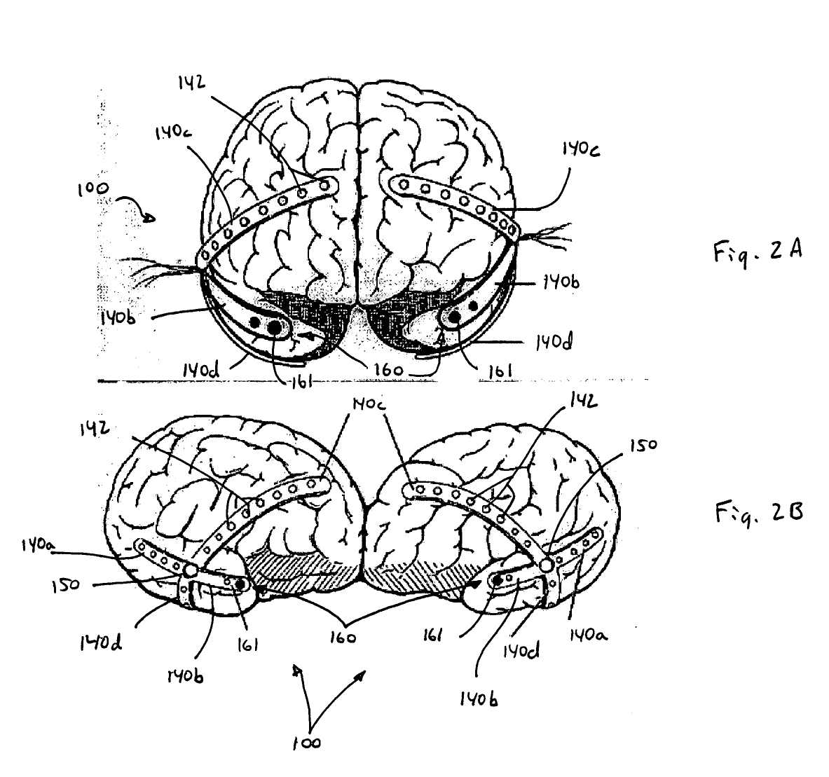

FIGS. 2A-2B illustrate a particular implementation of the system 100. For patients in whom the seizure focus involves structures including and/or beyond the medial temporal lobe (i.e., lateral temporal and frontal, parietal and/or occipital lobes), four signal delivery devices 140a-140d (e.g., subdural strip electrodes or other arrangements) may be placed through each burr hole 150 (single or multiple, in unilateral or bilateral temporal and/or extratemporal regions) for therapeutic electrical cortical stimulation. FIGS. 2A and 2B illustrate such an arrangement of signal delivery devices 140a-140d placed over both the left and right temporal lobes. In other embodiments, the signal delivery devices can have other arrangements. For example, an electrical signal delivery device in accordance with one embodiment can include a substrate or support member having a two-dimensional grid or array of electrodes or electrode contacts.

The system 100 can also include one or more feedback devices 160 that provide an indication of the efficacy of the applied electrical signals. A practitioner can accordingly update the manner in which the signals are provided, based at least in part on the feedback, or the system can automatically update the signal delivery parameters based at least in part on this information. The feedback can be provided via a number of suitable modalities. For example, the signal delivery devices 140a-140d can include electrodes or contacts 142 that deliver electrical signals for therapeutic purposes, and (during interstitial periods), receive electrical signals from the brain for diagnostic purposes. In another arrangement, some of the electrical contacts 142 can be dedicated to a signal delivery function, and others can be dedicated to a diagnostic signal reception function (e.g., an EEG signal reception function). In other embodiments, other techniques can be used to measure brain activity and provide appropriate feedback. Suitable techniques can include flowmetry techniques (e.g., NADH fluorescence), cerebral blood flow monitoring or redox monitoring (e.g., metabolic monitoring) and/or event-related potential (ERP) monitoring. Therapeutic cortical stimulation inhibiting epileptogenicity may also be tailored based on premonitory (e.g., pre-ictal) evidence of subdural EEG-detected chaos changes. In any of these embodiments, the feedback device 160 can be located subdurally for improved sensitivity to brain activity. The feedback device can be carried by the signal delivery devices 140a-140d, or it can be a separate unit. For example, the feedback device 160 can include one or more of the contacts 142, as discussed above, or it can include a cerebral blood flow (CBF) probe 161. For purposes of illustration, a single CBF probe 161 is shown in FIGS. 2A-2B for each brain hemisphere, but the system 100 can include multiple CBF probes 161 (or other diagnostic devices) depending on factors that include the number and spatial distribution of the signal delivery contacts 142.

In still further embodiments, other feedback techniques can be used in addition to or in lieu of the foregoing subdural techniques. Such techniques can include monitoring the patient using fMRI or PET techniques. While it is expected that the subdural techniques will provide more effective feedback, these other techniques may be suitable in cases where subdural techniques are not practicable.

Aspects of the invention are expected to cure or at least alleviate symptoms of medically intractable temporal lobe epilepsy and its associated dysfunctions or disorders. As discussed above, such disorders can include neurological, psychological and/or neuropsychiatric disorders (i.e., interictal behavior syndrome disorder of temporal lobe epilepsy, the temporal lobe syndrome, Epilepsy Spectrum Disorder (ESD), the psychiatric symptoms characteristic of the neurobehavioral syndrome of epilepsy (i.e., Morel syndrome) and Geschwind syndrome). As was also discussed above, electrical signals delivered in accordance with particular embodiments may improve or enhance normal cerebral and/or mental functioning. Additional benefits of cortical stimulation, beyond seizure control, can include treatment of neuropsychological and/or sensory-motor disorders using subdural cortical stimulation in diseases which may be causative for, associated with, or a result of epilepsy involving motor, sensory, visual, pain perception, receptive and/or expressive language, memory, olfactory, gustatory and/or auditory dysfunction and neuropsychological or psychiatric disorders such as depression, obsessive-compulsive disorder, rage attacks, anxiety disorders and disassociative/ experiential states and disorders, depending upon the location of cerebral cortical dysfunction and the region of cerebral cortex amenable to therapeutic electrical stimulation and/or neuromodulation.

In particular embodiments, techniques similar (at least in part) to those described above in the context of treating epilepsy and associated neurological and neuropsychological disorders can enhance normal (and/or above normal) functioning of the brain, including accentuating complex verbal and/or nonverbal problem-solving abilities, memory, language, stroke recovery, tinnitus recovery, visual, sensory, motor, auditory and olfactory function. Accordingly, embodiments of the foregoing devices may be implanted in a reversible manner, operate in a nondestructive manner, and may be tailored individually to meet any given patient's therapeutic needs.

FIG. 3 is a flow diagram illustrating a representative process 300 for treating a patient in accordance with several embodiments of the invention. In other embodiments, aspects of the illustrated processes may be changed or eliminated, depending upon particular patient needs. Process portion 301 includes identifying a target cortical neural site associated with a function of a patient, which the patient performs at normal or better levels. For example, the patient may perform memory tasks at normal or above normal levels, but may wish to further enhance memory performance. Accordingly, identifying the target cortical neural site can include using an imaging technique (e.g., fMRI or PET techniques) to identify areas of the brain that are active when the patient performs a memory task. In other embodiments, the target cortical neural site can be determined in other manners, for example via reference to known cortical functions performed by cortical structures that are identified with reference to known anatomical landmarks.

Based upon the information obtained in process portion 301, the practitioner can implant an electrical signal delivery device at a subdural location proximate to the target cortical neural site (process portion 302). In process portion 303, the function of the patient is improved by applying electrical signals to the target neural site via the electrical signal delivery device on a generally continual basis at a frequency of from about 0.9 Hz to about 250 Hz. Optionally, the electrical signals may be applied in conjunction with an adjunctive behavior, as part of an overall treatment regimen. For example, if the target function to be improved is the performance of memory tasks, the patient can perform memory exercises at the same time as the patient receives the electrical signals. If the target function is mathematical problem solving, or musical performance, or language performance (e.g., learning a new language), the adjunctive therapy can include solving mathematical problems, or playing a musical instrument, or performing a language-based task, respectively. In further particular embodiments, the characteristics of the electrical signals may be different when the patient performs the adjunctive behavior than at other times, but in other embodiments, the signal characteristics can remain the same whether the patient performs the adjunctive behavior or not.

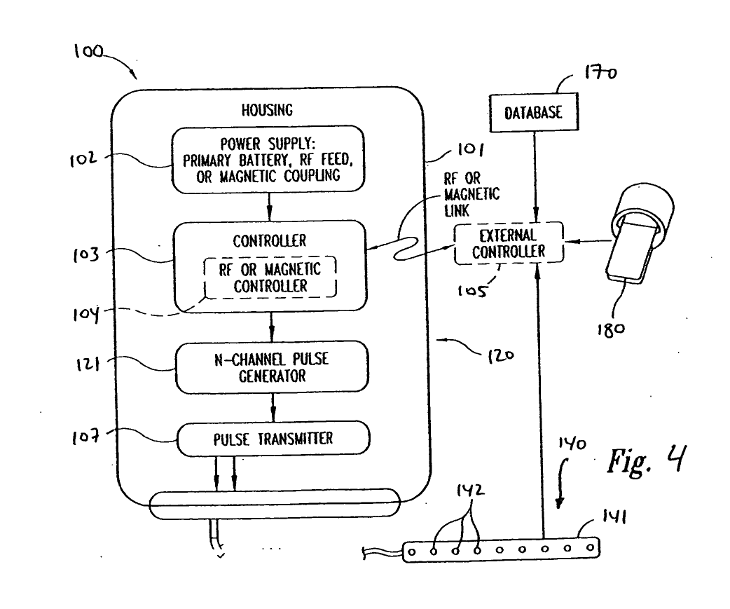

Process portion 304 includes receiving automated patient feedback, corresponding to the target function. For example, process portion 304 can include receiving feedback from implanted monitoring devices (e.g., implanted cerebral blood flow monitors) that identify active areas of the brain during the course of therapy. In at least some cases, the location at which the signals are provided can be changed (process portion 305) based at least in part on the automated feedback. For example, if it appears that the area of the brain most active when the patient performs a memory task is not located proximate to the electrode or electrodes that are applying the electrical signals, and that other electrodes are more proximate to this area, the practitioner can remotely change the signals so that they are applied by the electrodes closest to the appropriate target area. In other embodiments, other signal parameters can be changed in addition to or in lieu of the location at which the signals are provided. Further details of devices that may be used in accordance with the foregoing methods are described below with reference to FIGS. 4 and 5.

FIG. 4 schematically illustrates details of an embodiment of the system 100 described above. The overall system 100 includes the pulse system 120, at least a portion of which is carried by a housing 101. Accordingly, the housing 101 can carry a power supply 102, an integrated controller 103, a pulse generator 121, and a pulse transmitter 107. In certain embodiments, a portion of the housing 101 may comprise a signal return electrode. The power supply 102 can include a primary battery, such as a rechargeable battery, or other suitable device for storing electrical energy (e.g., a capacitor or supercapacitor). In other embodiments, the power supply 102 can be an RF transducer or a magnetic transducer that receives broadcast energy emitted from an external power source and that converts the broadcast energy into power for the electrical components of the system 100.

In one embodiment, the integrated controller 103 can include a processor, a memory, and/or a programmable computer medium. The integrated controller 103, for example, can be a microcomputer, and the programmable computer medium can include software loaded into the memory of the computer, and/or hardware that performs the requisite control functions. In another embodiment, identified by dashed lines in FIG. 4, the integrated controller 103 can include an integrated RF or magnetic controller 104 that communicates with the external controller 105 via an RF or magnetic link. In such an embodiment, many of the functions performed by the integrated controller 103 may be resident on the external controller 105, and the integrated portion 104 of the integrated controller 103 may include a wireless communication system.

The integrated controller 103 is operatively coupled to, and provides control signals to, the pulse generator 121, which may include a plurality of channels that send appropriate electrical pulses to the pulse transmitter 107. The pulse transmitter 107 is coupled to electrodes or contacts 142 carried by an electrode device 141 or other signal delivery device. In one embodiment, each of these contacts 142 is configured to be physically connected to a separate lead, allowing each contact 142 to communicate with the pulse generator 121 via a dedicated channel. Accordingly, the pulse generator 121 may have multiple channels, with at least one channel associated with each of the contacts 142 described above. Suitable components for the power supply 102, the integrated controller 103, the external controller 105, the pulse generator 121, and the pulse transmitter 107 are known to persons skilled in the art of implantable medical devices.

The pulse system 120 can be programmed and operated to adjust a wide variety of stimulation parameters, for example, which contacts 142 are active and inactive, whether electrical stimulation is provided in a unipolar or bipolar manner, and/or how stimulation signals are varied. In particular embodiments, the pulse system 120 can be used to control the polarity, frequency, duty cycle, amplitude, and/or spatial and/or topographical qualities of the stimulation. At certain times during a treatment regimen, the stimulation can be varied to match, approximate, or simulate naturally occurring burst patterns (e.g., theta-burst and/or other types of burst stimulation), and/or the stimulation can be varied in a predetermined, pseudorandom, and/or other aperiodic manner at one or more times and/or locations.

In particular embodiments, the pulse system 120 can receive information from selected sources, with the information being provided to influence the time and/or manner by which the signal delivery parameters are varied. For example, the pulse system 120 can communicate with a database 170 that includes information corresponding to reference or target functional performance values. Sensors can be coupled to the patient to provide measured or actual values corresponding to one or more parameters. The measured values of the parameter can be compared with the target value of the same parameter. Accordingly, this arrangement can be used in a closed-loop fashion to control aspects of the electrical signals. In one embodiment, some contacts 142 may deliver electromagnetic signals to the patient while others are used to sense the activity level of a neural population, as described above. In other embodiments, the same contacts 142 can alternate between sensing activity levels and delivering electrical signals, as was also described above. In either embodiment, information received from the signal delivery device 140 (or other devices) can be used to determine the effectiveness of a given set of signal parameters and, based upon this information, can be used to update the signal delivery parameters. This information can accordingly be used to determine which contacts 142 to activate. For example, if it appears that a particular area of the brain is active when the patient performs a target function (e.g., a memory task, a mathematical task, or a musical task), the contact 142 closest to that area can be activated to enhance the patient's level of functioning. This information can also be used to vary other signal delivery parameters (e.g., waveform, frequency, current and/or pulse width).

In other embodiments, other techniques can be used to provide patient-specific feedback. For example, a magnetic resonance chamber 180 can provide information corresponding to the locations at which a particular type of brain activity is occurring and/or the level of functioning at these locations, and can be used to identify additional locations and/or additional parameters in accordance with which electrical signals can be provided to the patient to further increase functionality. Accordingly, the system can include a direction component configured to direct a change in an electromagnetic signal applied to the patient's brain based at least in part on an indication received from one or more sources.

In still further embodiments, other techniques are used to provide patient-specific feedback. For example, the practitioner may implant cerebral blood flow monitors either as part of the signal delivery device (as shown in FIGS. 2A and 2B) or as separate units, to monitor local brain activity. Feedback from the cerebral blood flow monitors or other devices can then be used in any of the manners described above to control the delivery of electrical signals to selected electrodes.

FIG. 5 is a top, partially hidden isometric view of another embodiment of a signal delivery device 540, configured to carry multiple cortical electrodes or electrode contacts 542. The contacts 542 can be carried by a flexible support member 544 to place each contact 542 in contact with a stimulation site of the patient when the support member 544 is implanted. Electrical signals can be transmitted to the contacts 542 via leads carried in a communication link 545. The communication link 545 can include a cable 546 that is connected to the pulse system 120 (FIG. 4) via a connector 547, and is protected with a protective sleeve 548. Coupling apertures or holes 549 can facilitate attachment of the signal delivery device 540 to the patient at, or at least proximate to, a stimulation site, and beneath the dura mater. The contacts 542 can be biased cathodally and/or anodally. In an embodiment shown in FIG. 5, the signal delivery device 540 can include six contacts 542 arranged in a 2×3 electrode array (i.e., two rows of three electrodes each), and in other embodiments, the signal delivery device 540 can include more or fewer contacts 542 arranged in symmetrical or asymmetrical arrays. The particular arrangement of the contacts 542 can be selected based on the region of the patient's brain that is to be stimulated, and/or the patient's condition.

Several of the foregoing embodiments can provide advantages over existing systems. For example, systems that have exclusively subdural (but not deep brain) electrical contacts, can be less invasive than existing systems that use deep brain electrodes. Applying electrical signals subdurally is also expected to provide more effective and more efficient treatment to the patient. The treatment can be used to address epileptogenicity, non-epileptogenic disorders (e.g., stroke and/or tinnitus), and/or functions performed at normal or better than normal levels by the patient. It is expected that in any of these cases, the generally continuous nature of the stimulation will provide enhanced therapeutic benefits to the patient. These benefits can further be enhanced by direct, targeted “on demand” stimulation to a different brain area, and/or in accordance with another change in signal delivery parameters. For example, the pulse system can provide continuous pulses to address interictal dysfunction, and can also provide targeted, ictal signals directed to addressing (e.g., disrupting or inhibiting) specific seizure events. In the context of non-epiletogenic treatment, the system can provide stimulation in accordance with different parameters (e.g., to a different brain location) when the patient engages in an adjunctive behavior than at other times.

From the foregoing, it will be appreciated that specific embodiments of the invention have been described herein for purposes of illustration, but that various modifications may be made without deviating from the invention. For example, the electrodes or contacts may have configurations other than those shown in the Figures (e.g., curved strip shapes, or grids with dimensions different than 2×3). The system can provide (or receive) signals in accordance with parameters and/or modalities other than those specifically identified above. Aspects of the invention described in the context of particular embodiments may be combined or eliminated in other embodiments. For example, aspects of the systems and methods described in the context of epileptogenicity may apply to non-epileptogenic treatments, and vice versa. Further, while advantages associated with certain embodiments of the invention have been described in the context of those embodiments, other embodiments may also exhibit such advantages, and not all embodiments need necessarily exhibit such advantages to fall within the scope of the invention. Accordingly, the invention is not limited, except as by the appended claims.

I claim:

1. A method for treating a patient, comprising:
identifying a target neural site at a parahippocampal gyrus of a patient;
implanting an electrode at a subdural location at least proximate to the target neural site; and
at least reducing both ictal and interictal epileptogenicity of the patient, including interictal neural dysfunction associated with epileptogenicity, by applying electrical signals to the target neural site via the electrode on a generally continual basis both ictally and interictally at a frequency of from about 0.9 Hz to about 250 Hz.

2. The method of claim 1 wherein applying electrical signals includes applying electrical signals at a current in the range of from about 0.1 mA to about 10 mA, and a pulse width in the range of from about 0.025 ms to about 0.5 ms.

3. The method of claim 1 wherein at least reducing interictal neural dysfunction associated with epileptogenicity includes at least reducing effects of a neuropsychological or neuropsychiatric disorder.

4. The method of claim 3 wherein at least reducing interictal neural dysfunction associated with epileptogenicity includes at least reducing effects of interictal behavior syndrome disorder.

5. The method of claim 3 wherein at least reducing effects of a neuropsychological or neuropsychiatric disorder includes at least reducing the effects of depression, an obsessive-compulsive disorder, rage attacks, an anxiety disorder, a disassociative disorder, or an experiential disorder.

6. The method of claim 3 wherein at least reducing effects of a neuropsychological or neuropsychiatric disorder includes at least reducing the effects of psychosis, delusional disorders, mania, personality disorders, or Geschwind syndrome.

7. The method of claim 1 wherein at least reducing interictal neural dysfunction associated with epileptogenicity includes at least reducing effects on at least one of the patient's memory, learning, behavior, mood and senses.

8. The method of claim 1 wherein the electrical signals are first electrical signals, and wherein the method further comprises providing second electrical signals in direct response to epileptic seizure activity.

9. A method for treating a patient, comprising:
implanting an electrical signal delivery device proximate to a target neural site located at a temporal lobe of a patient; and
at least reducing non-epileptogenic symptoms of the patient by applying electrical signals to the target neural site via the electrical signal delivery device on a generally continual basis at a frequency of from about 0.9 Hz to about 250 Hz.

10. The method of claim 9 wherein at least reducing non-epileptogenic symptoms of the patient includes at least reducing symptoms associated with a stroke.

11. The method of claim 9 wherein at least reducing non-epileptogenic symptoms of the patient includes at least reducing symptoms associated with tinnitus.

12. The method of claim 9 wherein at least reducing non-epileptogenic symptoms includes at least reducing effects of a neuropsychological or neuropsychiatric disorder.

13. The method of claim 12 wherein at least reducing effects of a neuropsychological or neuropsychiatric disorder include at least reducing the effects of depression, an obsessive-compulsive disorder, rage attacks, an anxiety disorder, a disassociative disorder, or an experiential disorder.

14. The method of claim 12 wherein at least reducing effects of a neuropsychological or neuropsychiatric disorder include at least reducing the effects of psychosis, delusional disorders, mania, personality disorders, or Geschwind syndrome.

15. The method of claim 9 wherein at least reducing non-epileptogenic symptoms includes at least reducing effects on at least one of the patient's memory, learning, behavior, mood and senses.

16. A method for treating a patient, comprising:
implanting an electrical signal delivery device subdurally proximate to a target neural site at a cortical location of a patient; and
improving a non-epileptogenic neurological characteristic of the patient by applying electrical signals to the target neural site via the electrical signal delivery device on a generally continual basis at a frequency of from about 0.9 Hz to about 250 Hz.

17. The method of claim 16 wherein applying electrical signals includes applying electrical signals at a frequency of from about 0.9 Hz to about 130 Hz.

18. The method of claim 16 wherein applying electrical signals includes applying electrical signals at a frequency of from about 50 Hz to about 100 Hz.

19. The method of claim 16, further comprising identifying the target neural site via a functional imaging technique.

20. The method of claim 16 wherein implanting an electrical signal delivery device includes implanting an electrical signal delivery device having multiple electrical contacts, and wherein the method further comprises:
receiving a feedback signal from the patient indicative of neural activity;
identifying a target contact located at least proximate to the neural activity; and
wherein applying electrical signals includes applying electrical signals to the target contact.

21. The method of claim 16 wherein receiving a feedback signal includes receiving a feedback signal from an implanted device.

22. The method of claim 16 wherein improving a neurological characteristic of a patient includes improving a neurological characteristic of a normally-functioning patient.

23. The method of claim 16 wherein improving a neurological characteristic of the patient includes improving a neurological characteristic that is at normal or better levels.

24. The method of claim 23 wherein improving a neurological characteristic of the patient includes improving a memory function of the patient.

25. The method of claim 23 wherein improving a neurological characteristic of the patient includes improving a neuropsychological function of the patient.

26. The method of claim 16 wherein implanting the electrical signal delivery device includes implanting an electrode at a subdural location.

27. The method of claim 16 wherein applying electrical signals includes enhancing neural cell metabolism.

28. The method of claim 16 wherein applying electrical signals includes applying electrical signals continuously over a period of several months.

29. The method of claim 16 wherein applying electrical signals includes applying electrical signals continuously over a period of several years.

30. The method of claim 16 wherein applying electrical signals includes applying electromagnetic signals at a subthreshold level.

31. The method of claim 16 wherein applying electrical signals includes applying electrical signals to increase cerebral blood flow to the target neural site.

32. The method of claim 16 wherein implanting the electrical signal device includes implanting a strip-shaped support member having multiple electrical contacts positioned within the patient's skull.

33. The method of claim 16 wherein implanting an electrical signal device includes implanting an electrical signal device at a temporal lobe of the patient.

34. The method of claim 16, further comprising directing the patient to engage in an adjunctive behavior as part of a treatment regimen that includes both the adjunctive behavior and the application of electrical signals.

35. The method of claim 34 wherein directing the patient to engage in an adjunctive behavior includes directing the patient to engage in an adjunctive behavior simultaneously with applying the electrical signals.

36. The method of claim 34 wherein directing the patient to engage in an adjunctive behavior includes directing the patient to engage in at least one of a language task, a memory task, a musical task and a mathematical task.

37. A method for treating a patient, comprising:
identifying a target cortical neural site associated with a function that a patient performs at normal or better levels;
implanting an electrical signal delivery device at a subdural location proximate to the target cortical neural site;
improving the function of the patient by applying electrical signals to the target neural site via the electrical signal delivery device on a generally continual basis at a frequency of from about 0.9 Hz to about 250 Hz;
receiving automated feedback from the patient corresponding to the function; and
changing a location at which the signals are provided, based at least in part on the automated feedback.

38. The method of claim 37 wherein the electrical signal delivery device includes multiple electrical contacts and wherein changing a location at which the signals are provided includes changing a contact to which the signals are directed.

39. The method of claim 37 wherein improving a function includes improving a memory function.

40. The method of claim 37 wherein improving a function includes improving a learning function.

41. The method of claim 37 wherein receiving automated feedback includes receiving feedback from a feedback device implanted in the patient.

42. The method of claim 37, further comprising directing the patient to engage in an adjunctive behavior that includes a task directly associated with the function.

43. The method of claim 42 wherein directing the patient to engage in an adjunctive behavior includes directing the patient to engage in at least one of a language task, a memory task, a musical task and a mathematical task.
